(19) United States
(12) Patent Application Publication
(10) Pub. No.: US 2026/0116870 A1
(43) Pub. Date: Apr. 30, 2026

(54) NOVEL NAV 1.1 POTENTIATORS

(71) Applicant: Boehringer Ingelheim International GmbH, Ingelheim am Rhein (DE)

(72) Inventors: Kai GERLACH, Mittelbiberach (DE); Sarah Margaret MAJOR, Didcot (GB); Konstantinos RAMPALAKOS, Berkshire (GB)

(21) Appl. No.: 19/369,688

(22) Filed: Oct. 27, 2025

Related U.S. Application Data
(60) Provisional application No. 63/713,180, filed on Oct. 29, 2024.

(51) Int. Cl. C07D 401/14; A61K 31/4439

(52) U.S. Cl. CPC C07D 401/14; A61K 31/4439

(57) ABSTRACT

The present invention relates to 2,3-dihydro-1H-isoindol-1-one derivatives, their pharmaceutical compositions containing them and their use in therapy, particularly in the treatment or prevention of conditions having an association with the voltage-gated sodium channel Naᵥ1.1.

### FIELD OF THE INVENTION

The present invention relates to 2,3-dihydro-1H-isoindol-1-one derivatives, their pharmaceutical compositions containing them and their use in therapy, particularly in the treatment or prevention of conditions having an association with the voltage-gated sodium channel Naᵥ1.1.

### BACKGROUND OF THE INVENTION

Nav1.1 is a voltage-gated sodium channel (NaV) which is formed by the alpha-subunit encoded by SCN1A, as well as a regulatory subunit encoded by SCN1B. Nav1.1 is predominantly expressed in the brain (Catterall, W. A., J Physiol 2012, 590, 2577-2589, and Catterall, W. A., Neurochem Res 2017, 42, 2495-2504). Furthermore, Nav1.1 is largely expressed in parvalbuminpositive fast spiking GABAergic interneurons (PVs) and there regulates membrane depolarization and action potential (AP) firing (Ogiwara, I. et al., J Neurosci 2007, 27, 5903-5914). Loss of Nav1.1 channel function leads decreased firing of PVINs, leading to a disinhibition, and hence overexcitation, of excitatory pyramidal neurons, which is known to be associated with numerous mental health disorders. (Han, S. et al., Nature 2012, 489, 385-390, Oakley, J. C. et al., Epilepsia 2011, 52(Suppl. 2), 59-61, and Verret, L. et al., Cell 2012, 149, 708-721). Preclinical and clinical findings suggest that the excitatory/inhibitory balance (E/I-balance) in the prefrontal cortex is crucial for cognitive control/flexibility (Lewis et al., 2012, Weinstein et al, 2017). The E/I balance is formed by a prefrontal micro-circuit between the PV interneurons and glutamatergic pyramidal neurons, which generates a specific neural network activity responsible for the formation of γ-oscillations. In this microcircuit, PV interneurons are known to play a critical role in the entrainment of γ-oscillations (Toader et al. 2020). Patients with Schizophrenia have disturbed E/I balance as determined by robust γ-oscillation deficits (Hirano et al., 2015; White & Siegel, 2016; Light et al., 2020). Furthermore, patients with schizophrenia show impairments in cognitive flexibility (Pantelis et al., 2009) which correlate with the γ-oscillation deficits (Light et al. 2020). Furthermore, Dravet syndrome is a rare genetic epileptic encephalopathy where more than 70% of patients have de novo heterozygous mutations of the SCN1A gene (Catterall, WA., Ann Rev Pharmacol Toxicol 2014, 54, 317-338), which result in a loss of function of NaV1.1 (Mantegazza, M. et al., Proc Natl Acacl Sci USA 2005, 102, 18177-18182).

Therefore, brain penetrant Nav1.1 activators should hold therapeutic potential to treat cognitive deficits associated with an E/I imbalance by lowering the action potential threshold on PV interneurons, thereby enhancing the phasic fast synaptic excitation of the PV interneurons.

Therefore, activation of Nav1.1 should restore the E/I balance and γ-oscillation function in the prefrontal cortex of patients to improve cognitive control and flexibility.

Only few voltage-gated sodium channel Naᵥ 1.1 potentiators have been reported in literature:
1) 2-Methyl benzamide derivatives [F. Crestey, K. Frederiksen, H. S. Jensen, K. Dekermendjian, P. H. Larsen, J. F. Bastlund, D. Lu, H. Liu, C. R. Yang, M. Grunnet, N. Svenstrup ACS Chem. Neurosci. 2015, 6, 1302-1308]
2) Thiophene carboxamide AA43279 [K. Frederiksen, D. Lu, J. Yang, H. S. Jensen, J. F. Bastlund, P. H. Larsen, H. Liu, F. Crestey, K. Dekermendjian, L. Badolo, M. Laursen, C. Hougaard, C. Yang, N. Svenstrup, M. Grunnet Eur. J. Neurosci. 2017, 46, 1887-1896]
3) Compound Lu AE98134 [N. L. von Schoubye, K. Frederiksen, U. Kristiansen, A. V. Petersen, N. O. Dalby, M. Grunnet, H. S. Jensen, T. Jespersen, V. S. Sohal, J.-F. Perrier Neurosci. Lett. 2018, 662, 29-35]
4) Phenyl-substituted nicotinamide derivatives [T. Miyazaki, M. Kawasaki, A. Suzuki, Y. Ito, A. Imanishi, T. Maru, T. Kawamoto, T. Koike Bioorg. Med. Chem. Lett. 2019, 29, 815-820].
   While significant advances have been made in this field, there remains a substantial need for compounds which are NaV1.1 potentiators, thereby being useful in treating cognitive dysfunctions associated with the E/I balance disruption, such as the cognitive impairments associated with schizophrenia (CIAS). Additionally, Nav1.1 potentiators are envisioned to treat seizure disorders, such as epilepsy, in a mammal, preferably a human.

### DETAILED DESCRIPTION OF THE INVENTION

The present invention provides novel 2,3-dihydro-1H-isoindol-1-one derivatives, namely the compounds 5-(1-tert-butyl-1H-pyrazol-4-yl)-7-(3-hydroxypropoxy)-2-[(6-methylpyridin-3-yl)methyl]-2,3-dihydro-1H-isoindol-1-one, 5-(1-tert-butyl-1H-pyrazol-4-yl)-7-(3-hydroxypropoxy)-2-[(6-methoxypyridin-3-yl)methyl]-2,3-dihydro-1H-isoindol-1-one, and 5-(1-tert-butyl-1H-pyrazol-4-yl)-7-(3-hydroxypropoxy)-2-[2-(pyrrolidin-1-yl)pyridin-4-yl]-2,3-dihydro-1H-isoindol-1-one:

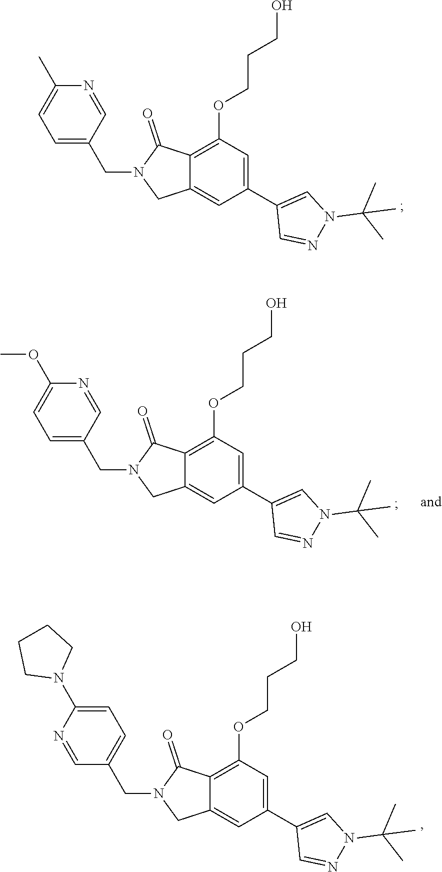

which are potent voltage-gated sodium channel Naᵥ 1.1 potentiators (assay A).

### General Definitions

Terms not specifically defined herein should be given the meanings that would be given to them by one skilled in the art considering the disclosure and the context.

### Stereochemistry:

Unless specifically indicated, throughout the specification and the appended claims, a given chemical formula or name shall encompass tautomers and all stereo, optical and geometrical isomers (e.g. enantiomers, diastereomers, E/Z isomers etc.) and racemates thereof, as well as mixtures in different proportions of the separate enantiomers, mixtures of diastereomers, or mixtures of any of the foregoing forms where such isomers and enantiomers exist, as well as salts, including pharmaceutically acceptable salts thereof.

### Salts:

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salts” refer to derivatives of the disclosed compounds wherein the parent compound forms a salt with an acid. Examples for acids forming a pharmaceutically acceptable salt with a parent compound containing a basic moiety include mineral or organic acids such as benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid, or tartaric acid.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof. Salts of other acids than those mentioned above which for example are useful for purifying or isolating the compounds of the present invention (e.g., trifluoroacetate salts) also comprise a part of the invention.

### Biological Assays

### Abbreviations

aq. aqueous
ACN acetonitrile
Ar argon
BSA bovine serum albumin
Cpd compound
CyHex cyclohexane
DCM dichloromethane
DMF N,N-dimethylformamide
DMSO dimethyl sulfoxide
EA ethyl acetate
EC₁₅₀ effective concentration at 150% effect
ESI-MS electrospray ionisation mass spectrometry
eq. equivalent(s)
h hour(s)
HEPES 4-(2-hydroxyethyl)-1-piperazineethanesulfonic acid
HPLC high-performance liquid chromatography
HPLC-MS high-performance liquid chromatography-mass spectrometry
HRMS high-resolution mass spectrometry
M molarity
MeOH methanol
min minutes
MS mass spectrometry
N Normality
NBS N-bromosuccinimide
NMP N-methyl-2-pyrrolidone
PE petroleum ether
RT room temperature
Rt retention time

The biological activity of compounds is determined by the following methods:

A. In Vitro Testing of Nav1.1 Potentiator: Automated Patch Clamp with Syncropatch 384PE

### 1) Syncropatch 384PE Platform and Software

The activity of voltage-dependent sodium (Naᵥ) channels and cpd-dependent modulation of Naᵥ channel activity were recorded with the automated patch clamp platform Syncropatch 384PE utilizing NPC-384 resistance chips (384-well format). Data acquisition was controlled with PatchControl384 software, and export and analysis were performed with DataControl384 software (all Nanion Technologies, Munich, Germany) and customised analysis software.

### 2) Buffers Used in the Experiments

The following extracellular solutions were utilized: Cell catch buffer containing (in mM) 140 NaCl, 4 KCl, 2 CaCl₂, 1 MgCl₂, 10 Glucose, 10 HEPES (pH adjusted to 7.4 with NaOH). Seal enhancer buffer containing (in mM) 80 NaCl, 3 KCl, 35 CaCl₂, 10 MgCl₂, 10 HEPES (pH adjusted to 7.4 with NaOH; cell catch buffer). Washing/run buffer containing (in mM) 140 NaCl, 4 KCl, 5 CaCl₂, 1 MgCl₂, 10 Glucose, 10 HEPES (pH adjusted to 7.4 with NaOH; cell catch buffer).

The intracellular solution was prepared by mixing to two buffers (90 vol % of buffer I with 10% of buffer II). Buffer I contained (in mM) 140 CsF, 10 NaCl, 1 EGTA, 10 HEPES and Buffer II contained (in mM) containing (in mM) 20 EDTA, 20 MgCl₂. After addition of 1 mM Na₂ATP, pH of the intracellular buffer was adjusted to 7.2 (with CsOH).

### 3) Cell Types

Experiments were performed utilizing HEK293 cells stably expressing SCN1A (NM_006920 (var 2); pD3.2 vector) and SCN1B (NM_001037.4; pD6.2 vector) encoding human Nav channel α subunit and β1 subunit, respectively (termed HEK-Nav1.1 cells; from SB DrugDiscovery).

### 4) Compound Plate Preparation:

Compound plates were prepared by Compound Logistic in Axygen deep well plates 384 (Thermo Fisher Scientific, Waltham, US), compound wells contained a volume of 300 nL. Each compound titration was prepared in quadruplicates in 100% DMSO. Starting from the highest concentration of 10 mM compounds were in diluted 1:3 on the plate to create a 11-point concentration-response curve. Compounds were then diluted by adding 50 μl/well of run buffer containing a 0.1% Pluronic F-127 solution. An extra 1:2 dilution was carried out by the Syncropatch during the experiments, reaching a final top concentration of 30 μM and 0.3% DMSO. Controls were manually prepared freshly on the day of the experiment. 0.3% DMSO was used as negative control and ATX 11 (30 nM in 0.3% DMSO) served as positive control.

### 5) Voltage Protocol, Data Sampling, Analysis, and Quality Control

From a holding potential of −100 mV, Naᵥ channels were activated by depolarizing voltage steps to 0 mV for 50 ms every 20 s. A total of 24 baseline sweeps and 17 sweeps after compound application were recorded, leak subtraction was applied in between sweeps. The following parameters were analysed: baseline (leak current), peak inward current, AUC (area under the curve), residual current after channel inactivation, TAU (time constant of inactivation), R², seal resistance, cell capacitance, Rₛ (series resistance). Recordings were only analysed, if current amplitude before application of cpd was >−200 pA, and if Rₛ was 30 MΩ through-out recordings. Recordings were discarded, if the following quality control criteria were not met: Residual current >50 pA, baseline current <−100 pA.

### Biological Data

TABLE 1
Biological data of the compounds of the present invention as determined in Assay A

| | | Assay A |
|---|---|---|
| | | Nav 1.1 Potentiator |
| Example | Structure | EC₁₅₀ [nM] |
| 1 | 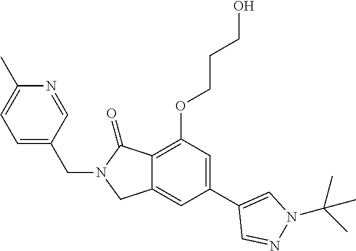 [SMILES: Cc1ccc(CN2Cc3cc(-c4cnn(C(C)(C)C)c4)cc(OCCCO)c3C2=O)cn1] | 51.1 |
| 2 | 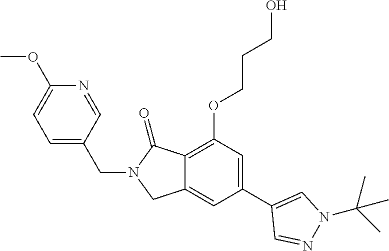 [SMILES: COc1ccc(CN2Cc3cc(-c4cnn(C(C)(C)C)c4)cc(OCCCO)c3C2=O)cn1] | 43.7 |
| 3 | 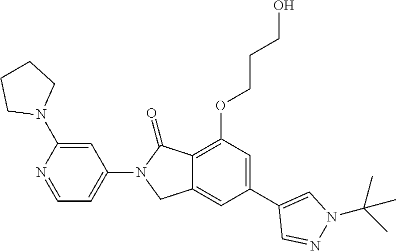 [SMILES: CC(C)(C)n1cc(-c2cc3c(c(OCCCO)c2)C(=O)N(c2ccnc(N4CCCC4)c2)C3)cn1] | 4440.0 |

This data demonstrates that compounds of the present invention are active Nav 1.1 potentiators.

### Use in Treatment/Method of Use

The present invention is directed to a compound which is useful in the treatment of a disease, symptom, disorder, and condition wherein the potentiation of Nav1.1 is of therapeutic benefit, including but not limited to the treatment and/or prevention of psychiatric and neurological conditions and symptoms associated with E/I balance dysfunction, such as cognitive impairments, seizure disorders or disorders such as anxiety and depression.

In view of their pharmacological effect, compounds of the present invention are suitable for use in the treatment or prevention of a disease, condition or associated symptom selected from the list consisting of (1) cognitive impairments associated with schizophrenia (CIAS), schizophreniform disorder, treatment resistant schizophrenia, schizotypal disorder, autism spectrum disorders (ASD), bipolar disorder, Alzheimer Disease (AD), Parkinson Disease (PD) attention deficit hyperactivity disorder (ADHD), frontotemporal dementia, Lewy-body dementia, vascular dementia, post-stroke dementia or Creutzfeldt-Jakob disease;

(2) seizure disorders, such as epilepsy, Dravet syndrome, Angelman syndrome, frontal lobe epilepsy, myoclonic epilepsy, temporal lobe epilepsy, generalized epilepsy disorder, Rett syndrome, drug resistant epilepsy, stroke, myasthenia gravis, brain and meningeal infections like encephalomyelitis and meningitis.

(3) mood disorders such as depression, major depressive disorder, manic depression, borderline personality disorder, anxiety disorders such as generalized anxiety disorder, panic disorders, post-traumatic stress disorders.

In one embodiment, the present invention is directed to a method for the treatment of a psychiatric or neurological condition in a patient comprising administering a therapeutically effective amount of a compound of the invention, or a pharmaceutically acceptable salt thereof, to said patient. In another embodiment, the psychiatric or neurological condition to be treated is selected from the group consisting of cognitive impairments associated with schizophrenia, bipolar disease, and seizure disorders.

As used herein, unless otherwise noted, the terms “treating”, “treatment” shall include the management and care of human subjects or patients for the purpose of combating a disease, condition, symptom, or disorder and includes the administration of the compound of the present invention to prevent the onset of the symptoms or complications, alleviate the symptoms or complications, or eliminate the disease, symptom, condition, or disorder.

As used herein, unless otherwise noted, the term “prevention” shall include (a) reduction in the frequency of one or more symptoms; (b) reduction in the severity of one or more symptoms; (c) delay or avoidance of the development of additional symptoms; and/or (d) delay or avoidance of the development of the disorder or condition.

The applicable daily dose of compounds of the present invention may vary from 0.001 to 2000 mg.

The actual pharmaceutically effective amount or therapeutic dose will depend on factors known by those skilled in the art such as age and weight of the patient, route of administration and severity of disease. In any case, the drug substance is to be administered at a dose, and in a manner, which allows a pharmaceutically effective amount to be delivered that is appropriate to the patient's condition.

### Pharmaceutical Compositions

Suitable preparations for administering the compounds of the present invention will be apparent to those with ordinary skill in the art and include for example tablets, pills, capsules, suppositories, lozenges, troches, solutions, syrups, elixirs, sachets, injectables, inhalatives, powders. The content of the pharmaceutically active compound(s) may vary in the range from 0.1 to 95 wt.-%, preferably 5.0 to 90 wt.-% of the composition as a whole.

Suitable tablets may be obtained, for example, by mixing a compound of the present invention with known excipients, for example inert diluents, carriers, disintegrants, adjuvants, surfactants, binders and/or lubricants and pressing the resulting mixture to form tablets.

The compound described herein can be administered topically, orally, transdermally, rectally vaginally, parentally, intranasally, intrapulmonary, intraocularly, intracardially, intramuscularly, intravenously, intradermally, intraperitoneally, subcutaneously, intraspinally, sublingually, subcapsularly, intraarticularly, or by inhalation.

### Combination Therapy

Compounds according to the invention, are particularly suitable as a co-medication (i.e. combination) with currently prescribed antipsychotics to treat in addition to the positive symptoms associated with schizophrenia also the cognitive and/or negative symptoms. Therefore, the compounds of general formula (I) according to the invention may be used in conjunction with other active substances (i.e., combination partner), particularly for the treatment and/or prevention of the diseases and conditions mentioned above. Other active substances which are suitable for such combination may be selected in the group consisting of: BACE inhibitors; amyloid aggregation inhibitors; directly or indirectly acting neuroprotective and/or disease-modifying substances; anti-oxidants (e.g. vitamin E or ginkolide); anti-inflammatory substances (e.g. COX inhibitors, NSAIDs additionally or exclusively having Abeta lowering properties); HMG-CoA reductase inhibitors (statins); acetylcholinesterase inhibitors (e.g., donepezil, rivastigmine, tacrine, galantamine); NMDA receptor antagonists (e.g. memantine, ketamine, esketamine, NR2b antagonists); AMPA receptor agonists; AMPA receptor positive modulators, AMPAkines, monoamine receptor reuptake inhibitors, substances modulating the concentration or release of neurotransmitters; substances inducing the secretion of growth hormone (e.g., ibutamoren mesylate and capromorelin); CE-1 receptor antagonists or inverse agonists; antibiotics (e.g., minocyclin or rifampicin); PDE1, PDE2, PDE4, PDE5, PDE9, PDE10 inhibitors, GABAA receptor agonists or positive modulators, GABAA receptor inverse agonists, GABAA receptor antagonists, nicotinic receptor agonists or partial agonists or positive modulators, alpha4beta2 nicotinic receptor agonists, alpha7 nicotinic receptor agonists or partial agonists or positive modulators; Somatostatin receptor 4 agonists or partial agonists or positive modulators, histamine H3 antagonists, 5HT-4 agonists or partial agonists, 5HT-6 antagonists, alpha2-adrenoreceptor antagonists, calcium antagonists, muscarinic receptor M1 agonists or partial agonists or positive modulators, muscarinic receptor M2 antagonists, muscarinic receptor M4 agonists or partial agonists or positive modulators, muscarinic receptor M4 antagonists, metabotropic glutamate-receptor 1 positive modulators, metabotropic glutamate-receptor 2 positive modulators, metabotropic glutamate-receptor 3 positive modulators, metabotropic glutamate-receptor 5 positive modulators, glycine transporter 1 inhibitors, antidepressants, such as citalopram, fluoxetine, paroxetine, sertraline and trazodone; anxiolytics, such as lorazepam and oxazepam; antipsychotics, such as aripiprazole, asenapine, clozapine, iloperidone, haloperidol, olanzapine, paliperidone, quetiapine, risperidone, ziprasidone, lurasidone, lumateperone, brexpiprazole and cariprazine; mood-stabilizers such as lithium and valproate, and other substances that modulate receptors or enzymes in a manner such that the efficacy and/or safety of the compounds according to the invention is increased and/or unwanted side effects are reduced. The compounds according to the invention may also be used in combination with immunotherapies (e.g., active immunisation with Abeta or Tau or parts thereof or passive immunisation with humanised anti-Abeta or anti-Tau antibodies or nanobodies) for the treatment of the above-mentioned diseases and conditions.

### Experimental Section

### Abbreviations

aq. aqueous
ACN acetonitrile
Ar argon
BSA bovine serum albumin
CyHex cyclohexane
DCM dichloromethane
DMF N,N-dimethylformamide
DMSO dimethyl sulfoxide
EA, EtOAc ethyl acetate
ESI-MS electrospray ionisation mass spectrometry
eq. equivalent(s)
h hour(s)
HEPES 4-(2-hydroxyethyl)-1-piperazineethanesulfonic acid
HPLC high-performance liquid chromatography
HPLC-MS high-performance liquid chromatography-mass spectrometry
HRMS high-resolution mass spectrometry
K₂CO₃ potassium carbonate
L liter
M molarity
MeOH methanol
min minutes
MS mass spectrometry
N Normality
NBS N-bromosuccinimide
NMP N-methyl-2-pyrrolidone
PdCl₂dppf [1,1′-Bis(diphenylphosphino)ferrocene]dichloropalladium(II)
PE petroleum ether
RT room temperature
Rt retention time
sat. saturated
TEA Triethylamine
TFA trifluoroacetic acid
TH F tetrahydrofuran
Tsunoda reagent (Tributylphosphoranylidene)acetonitrile solution

### HPLC-Methods:

### Method B

| time | Vol. % water | | Flow |
|---|---|---|---|
| (min) | (incl. 0.1% TFA) | Vol. % ACN | [mL/min] |
| 0.00 | 95 | 5 | 1.5 |
| 1.30 | 0 | 100 | 1.5 |
| 1.50 | 0 | 100 | 1.5 |

Analytical column: Sunfire 018 (Waters) 2.5 μm; 3.0×30 mm; column temperature: 60° C.

### Method C

| time | Vol. % water | | Flow |
|---|---|---|---|
| (min) | (incl. 0.1% TFA) | Vol. % ACN | [mL/min] |
| 0.00 | 95 | 5 | 1.3 |
| 0.02 | 95 | 5 | 1.3 |
| 1.0 | 0 | 100 | 1.3 |
| 1.3 | 0 | 100 | 1.3 |

Analytical column: Xbridge BEH 018 (Waters) 2.5 μm; 2.1×30 mm; column temperature: 60° C.

### Method D

| time | Vol. % water | Vol. % ACN | Flow |
|---|---|---|---|
| (min) | (incl. 0.1% FA) | (incl. 0.1% FA) | [mL/min] |
| 0.00 | 95 | 5 | 0.6 |
| 5.30 | 0 | 100 | 0.6 |
| 6.00 | 0 | 100 | 0.6 |
| 6.02 | 95 | 5 | 0.6 |
| 7.20 | 95 | 5 | 0.6 |

Analytical column: Phenomenex Kinetex-XB C18 1.7 μm; 2.1×100 mm; column temperature: 40° C.

### Method E

| time | Vol. % water | Vol. % ACN | Flow |
|---|---|---|---|
| (min) | (incl. 0.1% FA) | (incl. 0.1% FA) | [mL/min] |
| 0.00 | 95 | 5 | 0.9 |
| 1.10 | 0 | 100 | 0.9 |
| 1.35 | 0 | 100 | 0.9 |
| 1.40 | 95 | 5 | 0.9 |
| 1.50 | 95 | 5 | 0.9 |

Analytical column: UPLCTM BEHTM C18 (Waters) 1.7 μm; 2.1×50 mm; column temperature: 40° C.

### Method F

| time | Vol. % water | | Flow |
|---|---|---|---|
| (min) | (incl. 2 mM NH₄HCO₃) pH 10 | Vol. % ACN | [mL/min] |
| 0.00 | 95 | 5 | 0.6 |
| 5.30 | 0 | 100 | 0.6 |
| 5.80 | 0 | 100 | 0.6 |
| 5.82 | 95 | 5 | 0.6 |
| 7.00 | 95 | 5 | 0.6 |

Analytical column: UPLCTM BEHTM C18 (Waters) 1.7 μm; 2.1×100 mm; column temperature: 55° C.

### Preparation of Compounds of the Present Invention

### Example 1

### 5-(1-tert-butyl-1H-pyrazol-4-yl)-7-(3-hydroxypropoxy)-2-[(6-methylpyridin-3-yl)methyl]-2,3-dihydro-1H-isoindol-1-one

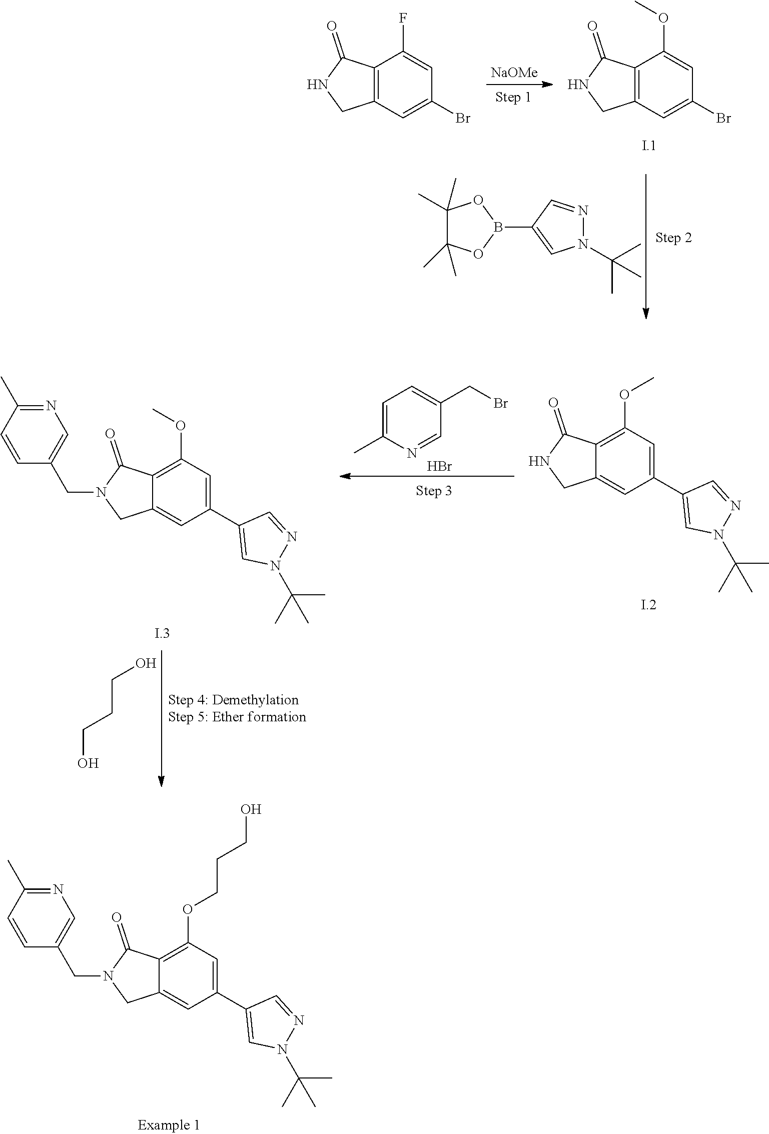

Step 1: 5-Bromo-7-fluoro-2,3-dihydro-1H-isoindol-1-one (5.00 g, 21.7 mmol) is dissolved in 50 mL methanol and stirred. Sodium methoxide (16 mL, 86.9 mmol) is added to the reaction and stirred overnight ab 40° C. The reaction is stopped, cooled and filtered to give 5-bromo-7-methoxy-2,3-dihydro-1H-isoindol-1-one (intermediate 1.1, 4.41 g, 17.9 mmol, 82.2% yield) as a pink solid.

| | |
|---|---|
| C9H8BrNO2 | (M = 242.0 g/mol) |
| ESI-MS: | 244 [M + H]+ |
| Rt (HPLC): | 1.97 min (method D) |

Step 2: 5-Bromo-7-methoxy-2,3-dihydro-1H-isoindol-1-one (Intermediate 1.1, 3.00 g, 12.4 mmol) 1-tert-butyl-4-(4,4,5,5-tetramethyl-1,3,2-dioxaborolan-2-yl)-1H-pyrazole (3.41 g, 13.6 mmol) and potassium carbonate (4.28 g, 31.0 mmol) are dissolved in 40 mL 1,4-dioxane and 8 mL water, and purged with nitrogen for 5 min. PdCl2dppf (0.45 g, 0.620 mmol) is added to the reaction mixture, purged for another 5 mins, sealed and stirred for 2 h at 100° C.

The reaction is cooled, filtered through celite and washed with EtOAc and water. The filtrate is diluted with water and the layers are separated. The aq. layer is extracted with EtOAc, the combined organic layers are washed with brine, dried over MgSO4, filtered, and concentrated to give the crude product.

The product is purified by flash column chromatography using 100% heptane-100% EtOAc to give 5-(1-tert-butyl-1H-pyrazol-4-yl)-7-methoxy-2,3-dihydro-1H-isoindol-1-one (intermediate 1.2, 2.50 g, 8.06 mmol, 65.04% yield) as a beige solid.

| | |
|---|---|
| C16H19N3O2 | (M = 285.3 g/mol) |
| ESI-MS: | 286 [M + H]+ |
| Rt (HPLC): | 0.67 min (method E) |

Step 3: To a solution of NaH (63 mg, 1.58 mmol) in 5 mL DMF is added under inert atmosphere a solution of 5-(1-tert-butyl-1H-pyrazol-4-yl)-7-methoxy-2,3-dihydro-1H-isoindol-1-one (intermediate 1.2, 300 mg, 1.05 mmol) in 5 mL DMF. The reaction mixture is stirred for 30 min at 65° C., then cooled to 0° C. A solution of 5-(bromomethyl)-2-methylpyridine hydrobromide (421 mg, 1.58 mmol) and NaH (63 mg, 1.58 mmol) in 10 mL DMF is added to the reaction mixture at 0° C. The reaction mixture is stirred for 2 h at 0° C.

The reaction is quenched with water an sat. Na2CO3 solution and is extracted with EtOAc. The combined organic phases are rinsed with water and brine, then dried over MgSO4, filtered and evaporated.

The product is purified by flash column chromatography using 0-100% 9/1 DCM-methanol in DCM to yield 5-(1-tert-butyl-1H-pyrazol-4-yl)-7-methoxy-2-[(6-methylpyridin-3-yl)methyl]-2,3-dihydro-1H-isoindol-1-one (intermediate 1.3, 275 mg, 0.690 mmol, 65.6% yield) as an off-white solid.

| | |
|---|---|
| C23H26N4O2 | (M = 390.4 g/mol) |
| ESI-MS: | 391 [M + H]+ |
| Rt (HPLC): | 1.72 min (method D) |

Step 4: To a solution of 5-(1-tert-butyl-1H-pyrazol-4-yl)-7-methoxy-2-[(6-methylpyridin-3-yl)methyl]-2,3-dihydro-1H-isoindol-1-one (Intermediate 1.3, 80 mg, 0.205 mmol) in 2 mL DCM is injected BBr3 (0.41 mL, 0.41 mmol) and stirred for 90 min at 0° C. under inert atmosphere. The reaction is quenched with 1 mL methanol, 2 mL water and 5 ml sat. solution of NaHCO₃ and then extracted with DCM. The combined organic phases are dried over MgSO4 and evaporated to give crude phenol which is taken forward directly into the next step.

| | |
|---|---|
| ESI-MS: | 377 [M + H]+ |
| Rt (HPLC): | 1.75 min (method D) |

Step 5: The residue is dissolved in 2 mL 1,4-dioxane treated with propane-1,3-diol (47 mg, 0.615 mmol), Tsunoda reagent (200 μl, 0.615 mmol) and then heated for 1 h at 50° C. The reaction is cooled to RT, quenched with 5 mL water and 5 mL sat. solution of NaHCO₃ and extracted with DCM. The combined organic phases are evaporated and purified by preparative HPLC (H2O/ACN/TFA) to give 5-(1-tert-butyl-1H-pyrazol-4-yl)-7-(3-hydroxypropoxy)-2-[(6-methylpyridin-3-yl)methyl]-2,3-dihydro-1H-isoindol-1-one (Example 1, 41 mg, 0.0925 mmol, 45.13% yield) as a colorless solid.

| | |
|---|---|
| C25H30N4O3 | (M = 435.5 g/mol) |
| ESI-MS: | 435 [M + H]⁺ |
| Rt (HPLC): | 1.81 min (method D) |

### Example 2

### 5-(1-tert-butyl-1H-pyrazol-4-yl)-7-(3-hydroxypropoxy)-2-[(6-methoxypyridin-3-yl)methyl]-2,3-dihydro-1H-isoindol-1-one

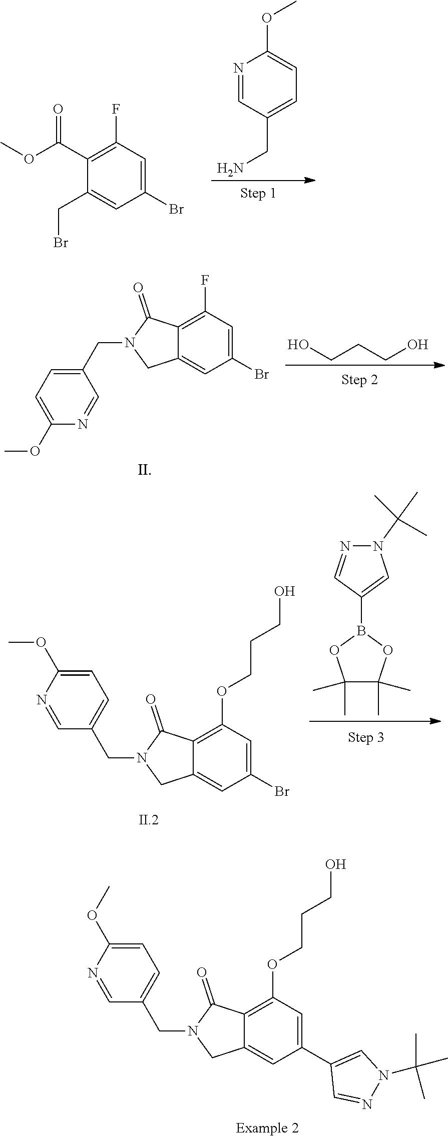

Step 1: To a solution of methyl 4-bromo-2-(bromomethyl)-6-fluorobenzoate (1.90 g, 4.95 mmol) in 15.1 ml ACN is added 1-(6-methoxypyridin-3-yl)methanamine (720 mg, 4.95 mmol), boric acid (307 mg, 4.95 mmol) and K2CO3 (2.05 g, 14.86 mmol). The reaction mixture is stirred at for 3 h 55° C. and overnight at RT.

The reaction is extracted with EtOAc and NaCl solution. The combined organic phases are dried over MgSO4 and evaporated.

The residue is diluted in methanol and ACN and is purified by preparative HPLC (H2O/ACN/TFA) to give 5-bromo-7-fluoro-2-[(6-methoxypyridin-3-yl)methyl]-2,3-dihydro-1H-isoindol-1-one (intermediate 11.1, 568 mg, 1.4 mmol, 27% yield) as a yellow solid.

| | |
|---|---|
| C15H12BrFN2O2 | (M = 351.1 g/mol) |
| ESI-MS: | 351 [M + H]⁺ |
| Rt (HPLC): | 0.65 min (method B) |

Step 2: To propane-1,3-diol (2.54 ml, 33.2 mmol) is added at 0° C. NaH (63.3 mg, 1.58 mmol). After 10 min is added 5-bromo-7-fluoro-2-[(6-methoxypyridin-3-yl)methyl]-2,3-dihydro-1H-isoindol-1-one (Intermediate 11.1, 277 mg, 0.79 mmol). The reaction mixture is stirred overnight at 65° C. The reaction is quenched with water and is extracted with DCM and water twice. The combined organic phases are dried over MgSO4 and evaporated. The residue is diluted in methanol and ACN and is purified by preparative HPLC (H2O/ACN/NH4OH) to give 5-bromo-7-(3-hydroxypropoxy)-2-[(6-methoxypyridin-3-yl)methyl]-2,3-dihydro-1H-isoindol-1-one (intermediate 11.2, 174 mg, 0.4 mmol, 54% yield) as a slightly yellow solid.

| | |
|---|---|
| C18H19BrN2O4 | (M = 407.2 g/mol) |
| ESI-MS: | 407 [M + H]⁺ |
| Rt (HPLC): | 0.57 min (method C) |

Step 3: To a solution of 5-bromo-7-(3-hydroxypropoxy)-2-[(6-methoxypyridin-3-yl)methyl]-2,3-dihydro-1H-isoindol-1-one (Intermediate 11.2, 1.00 g, 2.21 mmol) in 19.85 mL 1,4-dioxane is added under argon 1-tert-butyl-4-(4,4,5,5-tetramethyl-1,3,2-dioxaborolan-2-yl)-1H-pyrazole (663 mg, 2.65 mmol), potassium carbonate (2.2 mL, 4.42 mmol, 2M) and PdCl2dppf (0.90 g, 0.110 mmol). The reaction mixture is stirred for 4 h at 80° C. The reaction mixture is cooled to RT and stirred overnight. The reaction mixture is filtered and extracted with EtOAc and NaCl solution twice. The combined organic phases are dried over MgSO4 and evaporated. The residue is purified by column chromatography (silica gel; CH/(EtOAc/ethanol 50/50) 0-50% EtOAc/Ethanol). The residue is diluted in methanol and ACN and is purified by preparative HPLC (H2O/ACN/NH4OH) to give 5-(1-tert-butyl-1H-pyrazol-4-yl)-7-(3-hydroxypropoxy)-2-[(6-methoxypyridin-3-yl)methyl]-2,3-dihydro-1H-isoindol-1-one (Example 2, 667 mg, 1.48 mmol, 67% yield) as a white solid.

| | |
|---|---|
| C25H30N4O4 | (M = 450.5 g/mol) |
| ESI-MS: | 451 [M + H]⁺ |
| Rt (HPLC): | 0.61 min (method C) |

### Example 3

### 5-(1-tert-butyl-1H-pyrazol-4-yl)-7-(3-hydroxypropoxy)-2-[2-(pyrrolidin-1-yl)pyridin-4-yl]-2,3-dihydro-1H-isoindol-1-one

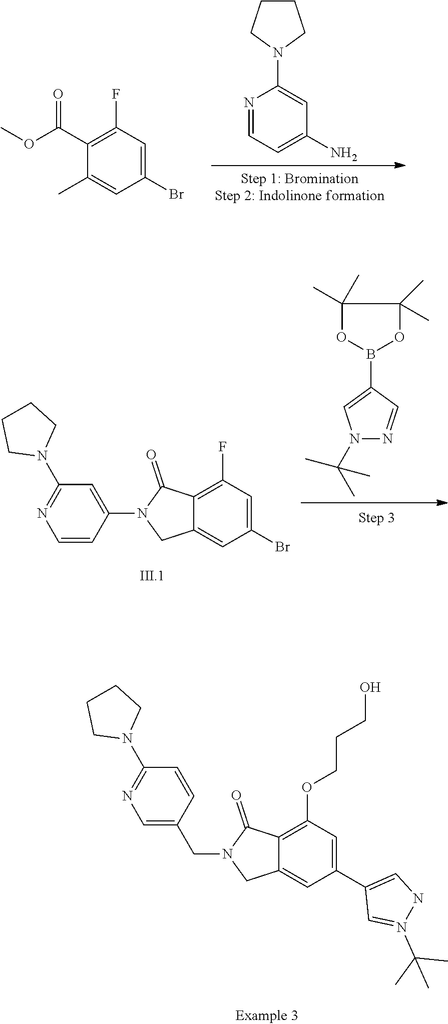

Step 1: To methyl 4-bromo-2-fluoro-6-methylbenzoate (200 mg, 0.81 mmol) is added benzoyl peroxide (42 mg, 0.12 mmol), N-bromo succinimide (173 mg, 0.971 mmol), ACN (5 mL) and the mixture is stirred for 4 h at 90° C. To the reaction mixture is added N-bromo succinimide (72 mg, 0.405 mmol) and the mixture is stirred overnight at 80° C. Formation of the intermediate benzyl bromide (not isolated) is observed: Rt (HPLC): 3.76 min (method F).

Step 2: To the reaction mixture containing the intermediate benzyl bromide is subsequently added 2-(pyrrolidin-1-yl)pyridin-4-amine (198 mg, 1.21 mmol), orthoboric acid (50 mg, 0.810 mmol), K2CO3 (112 mg, 0.810 mmol) and is stirred for 2 h at 80° C. The reaction is cooled to RT, filtered through a Whatman No1. Filter and the solvents are removed under reduced pressure. The residue is purified by column chromatography (silica gel; heptane/EtOAc 0-100% EtOAc followed by methanol/DCM 0-20% methanol) to give 5-bromo-7-fluoro-2-[2-(pyrrolidin-1-yl)pyridin-4-yl]-2,3-dihydro-1H-isoindol-1-one (intermediate 111.1, 39 mg, 0.104 mmol, 12.8% yield) as an off-white solid.

| | |
|---|---|
| C17H15BrFN3O | (M = 376.2 g/mol) |
| ESI-MS: | 377 [M + H]⁺ |
| Rt (HPLC): | 1.99 min (method D) |

Step 3: To NaH (8.3 mg, 0.207 mmol) was added a solution of propane-1,3-diol (15 μl, 0.207 mmol) in 1 mL 1,4-dioxane and is stirred for 10 min at RT. A solution of 5-bromo-7-fluoro-2-[2-(pyrrolidin-1-yl)pyridin-4-yl]-2,3-dihydro-1H-isoindol-1-one (intermediate 111.1, 39 mg, 0.104 mmol) in 1 mL 1,4-dioxane is added and the reaction mixture is stirred for 8 h at 60° C. under inert atmosphere. The reaction is cooled to RT and then carefully quenched with degassed water, treated with 1-tert-butyl-4-(4,4,5,5-tetramethyl-1,3,2-dioxaborolan-2-yl)-1H-pyrazole (313 mg, 1.25 mmol), K2CO3 (28 mg, 0.203 mmol) and PdCl2dppf (7.0 mg, 0.00858 mmol). The reaction mixture is stirred for 2 h at 80° C. under inert atmosphere.

The reaction mixture is cooled to RT and the solvents are evaporated. The residue is diluted with EtOAc and extracted with water twice. The combined organic phases are dried over MgSO4 and evaporated.

The residue is purified by preparative HPLC (H2O/ACN/NH4OH). The residue purified by preparative HPLC (H2O/ACN/TFA) to give 5-(1-tert-butyl-1H-pyrazol-4-yl)-7-(3-hydroxypropoxy)-2-[2-(pyrrolidin-1-yl)pyridin-4-yl]-2,3-dihydro-1H-isoindol-1-one (Example 3, 2.0 mg, 0.00400 mmol, 3.85% yield) as a white solid.

| | |
|---|---|
| C27H33N5O3 | (M = 475.5 g/mol) |
| ESI-MS: | 376 [M + H]⁺ |
| Rt (HPLC): | 0.65 min (method E) |

1. A compound selected from the group consisting of 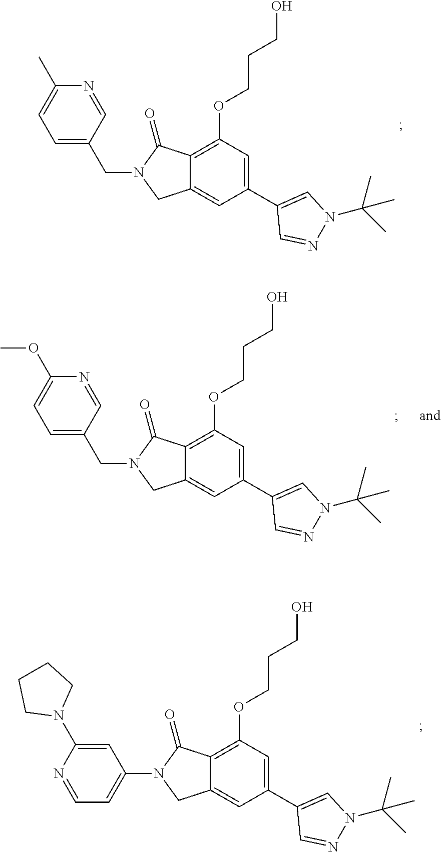
or a pharmaceutically acceptable salt thereof.

2. A compound according to claim 1, which is: 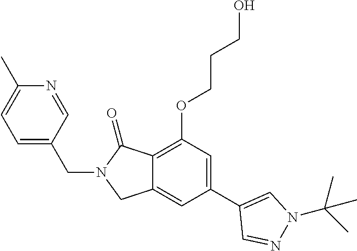 [SMILES: Cc1ccc(CN2Cc3cc(-c4cnn(C(C)(C)C)c4)cc(OCCCO)c3C2=O)cn1]
or a pharmaceutically acceptable salt thereof.

3. A compound according to claim 1, which is:

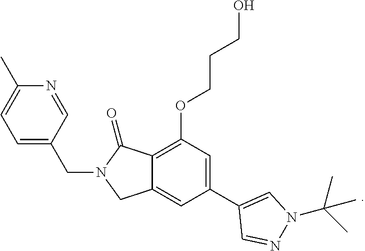 [SMILES: Cc1ccc(CN2Cc3cc(-c4cnn(C(C)(C)C)c4)cc(OCCCO)c3C2=O)cn1]

4. A compound according to claim 1, which is: 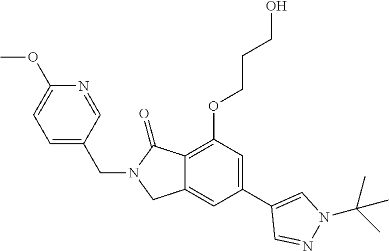 [SMILES: COc1ccc(CN2Cc3cc(-c4cnn(C(C)(C)C)c4)cc(OCCCO)c3C2=O)cn1]
or a pharmaceutically acceptable salt thereof.

5. A compound according to claim 1, which is:

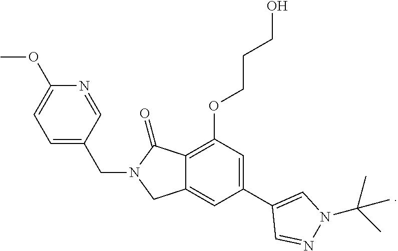 [SMILES: COc1ccc(CN2Cc3cc(-c4cnn(C(C)(C)C)c4)cc(OCCCO)c3C2=O)cn1]

6. A compound according to claim 1, which is: 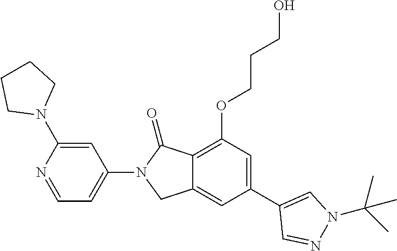 [SMILES: CC(C)(C)n1cc(-c2cc3c(c(OCCCO)c2)C(=O)N(c2ccnc(N4CCCC4)c2)C3)cn1]
or a pharmaceutically acceptable salt thereof.

7. A compound according to claim 1, which is:

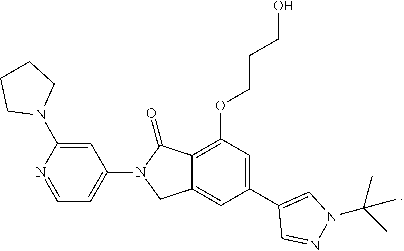 [SMILES: CC(C)(C)n1cc(-c2cc3c(c(OCCCO)c2)C(=O)N(c2ccnc(N4CCCC4)c2)C3)cn1]

8. A pharmaceutical composition comprising the compound according to claim 1 and a pharmaceutically acceptable adjuvant, diluent and/or carrier.

9. A method for the treatment of a psychiatric or neurological condition in a patient comprising administering a therapeutically effective amount of a compound according to claim 1, or a pharmaceutically acceptable salt thereof, to said patient.

10. A method according to claim 9, wherein the psychiatric or neurological condition to be treated is selected from the group consisting of cognitive impairments associated with schizophrenia, bipolar disease, and seizure disorders.
